(19) United States
(12) Patent Application Publication
(10) Pub. No.: US 2008/0058797 A1
(43) Pub. Date: Mar. 6, 2008

(54) UTERINE FOAM INSERT FOR ABLATION

(76) Inventor: Robert F. RIOUX, Ashland, MA (US)

Correspondence Address: FAY KAPLUN & MARCIN, LLP, 15O BROADWAY, SUITE 702, NEW YORK, NY, 10038, US

(21) Appl. No.: 11/745,681

(22) Filed: May 8, 2007

Related U.S. Application Data
(60) Provisional application No. 60/841,416, filed on Aug. 31, 2006.

(51) Int. Cl. A61B 18/18

(52) U.S. Cl. 606/41

(57) ABSTRACT

A system and method are described for tissue treating tissue within a hollow organ. The system may include a foam body having an operative shape adapted to fill at least a portion of the hollow organ, the foam body including a fluid retaining portion formed of a material selected to retain therein an electrically conductive fluid. The fluid retaining component includes at least one contacting portion which, when the foam body is inserted into the hollow organ in a desired position, contacts a target portion of tissue placing the fluid in contact with selected portions of an inner surface of the hollow organ when the foam body is in the operative shape.

### PRIORITY CLAIM

This application claims the priority to the U.S. Provisional Application Ser. No. 60/841,416, entitled “Uterine Foam Insert for Ablation,” filed Aug. 31, 2006. The specification of the above-identified application is incorporated herewith by reference.

### BACKGROUND INFORMATION

Disorders of the menstrual cycle including irregular bleeding, menhorrhagia, and dysfunctional uterine bleeding, among others, may result from an anatomic irregularity of the uterus, hormonal imbalance or systemic disease. The abnormal bleeding commonly results from fibroids, polyps or other abnormal growths within the uterus and treatment often involves procedures to remove this tissue.

Hysterectomy, or removal of the uterus, is often performed to provide long term relief to women who do not respond to medication or hormonal treatments. However, less invasive alternative procedures are being developed to treat these conditions to reduce side effects and shorten hospital stays and recovery periods. One such procedure is ablation of the lining of the uterus which involves removing portions of the endometrial lining including abnormal tissue that may be the source of the abnormal bleeding. In most common procedures, the endometrial lining is ablated using heat, electricity, a chemical application or cryogenic procedures. In these procedures, chemicals or energy are applied to target tissue until a desired level of treatment of the target tissue has been reached.

For example, the endometrial lining may be ablated by introducing heated saline solution into the uterus to contact target portions of the lining. The heated saline may be directed to a specific portion(s) of the uterine wall, or may be generally applied to the inner surface of the entire organ. However, free flowing fluids are indiscriminate ablating targeted and non-targeted parts of the lining and may leak from the uterus if complex procedures are not adhered to. Alternatively, heated fluids may be constrained within a conventional tube or a bag inserted into the uterus, to better determine on which tissue the fluid acts. However, in such systems heated fluids may not be able to reach all the targeted portions of the uterus leaving some target tissue untreated. In addition, with free and constrained saline systems it is difficult to obtain lesions of uniform depth which reach all the targeted tissue while minimizing damage to adjacent non-targeted tissue.

### SUMMARY OF INVENTION

The present invention relates to a system for tissue treating tissue within a hollow organ. The system may include a foam body having an operative shape adapted to fill at least a portion of the hollow organ, the foam body including a fluid retaining portion formed of a material selected to retain therein an electrically conductive fluid. The fluid retaining component includes at least one contacting portion which, when the foam body is inserted into the hollow organ in a desired position, contacts a target portion of tissue placing the fluid in contact with selected portions of an inner surface of the hollow organ when the foam body is in the operative shape.

The present invention also relates to a method for ablating tissue in a body cavity. A foam body in an insertion configuration is inserted into the body cavity. The foam body is expanded to an operative configuration within the body cavity so that the foam body substantially fills at least a portion of the cavity with a contacting portion of the foam body contacting a target portion of tissue to be ablated. The foam body is permeated with an electrically conductive fluid. Energy is transferred through the foam body to the contacting portion via the fluid to ablate the target tissue.

### BRIEF DESCRIPTION OF DRAWINGS

FIG. 1 is a diagram showing an embodiment of an uterine foam insert according to the invention;

FIG. 2 is a cross sectional view showing the embodiment according to the invention shown in FIG. 1, with a molded wire therein;

FIG. 3 is a pictorial representation of an experimental apparatus for testing an exemplary uterine foam insert according to the invention; and

FIG. 4 is a pictorial representation of test results derived from using the exemplary foam insert according to the invention, on animal tissue.

### DETAILED DESCRIPTION

The present invention may be further understood with reference to the following description and to the appended drawings, wherein like elements are referred to with the same reference numerals. The present invention relates to devices for ablating tissue within a hollow organ using RF energy.

Embodiments of the present invention are designed to overcome or minimize limitations of conventional ablation systems. According to the invention, a foam insert is inserted into the uterus, or in another body cavity to be treated, with an outer surface thereof in contact with target tissue. For example, a surface of the foam insert may be placed in contact with the endometrial lining. The foam insert may be shaped as desired to ensure contact with the target tissue while minimizing contact with non-targeted tissues. The foam insert may then be saturated with a fluid such as saline so that the fluid is in contact with those portions of target tissue which are in contact with an outer surface of the foam insert.

Once the foam insert and the fluid are in place within the body cavity, the fluid is used to convey energy to the target tissue. For example, a conventional RF generator (not shown) is electrically coupled to the foam insert to convey the RF energy thereto which is then conducted by the saline through the foam insert to the portions of target tissue contacting the foam insert to heat and ablate the target portions of the inner surfaces of the body cavity. As would be understood by those skilled in the art, a conventional RF generator may be used to generate the energy required to heat the tissue to achieve the desired degree of ablation. The transmission of energy from the external generator to the fluid may be enhanced or facilitated by the use of an energy receptor placed in the fluid or in the foam insert. For example, an antenna may be embedded in a body of the foam insert to more efficiently transfer RF energy to the fluid saturating the foam insert.

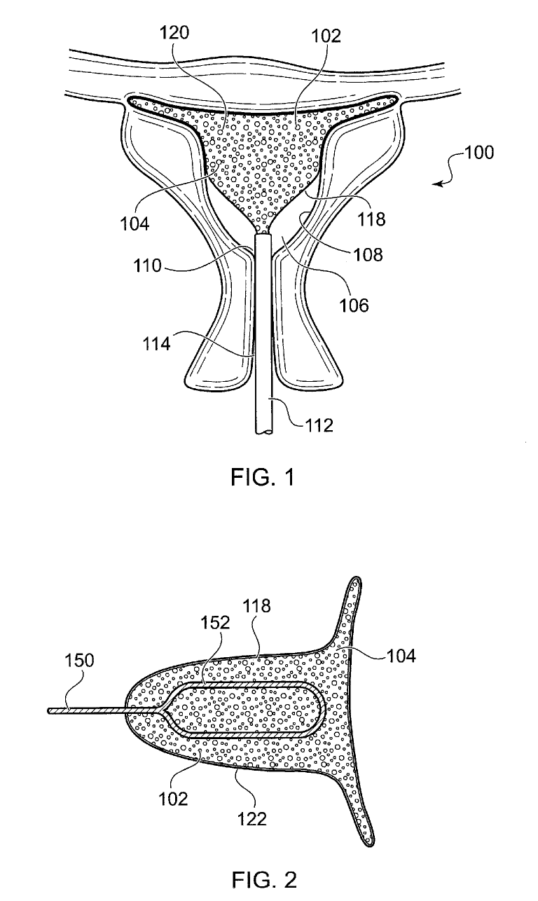

As shown in FIG. 1, an ablation system 100 according to the invention is specifically adapted designed to treat the endometrial lining of the uterus. However, those of skill in the art will understand that the present invention may be applied to transfer RF energy to portions of the inner linings of other body cavities and hollow organs in addition to the uterus for ablation or any other purpose. In the embodiment shown, a foam insert 102 is inserted into the uterus 106 through, for example, the cervical opening 110. The foam insert is formed completely or at least partially of a material 104 designed to retain a fluid 120 within the confines of the foam insert 102. For example, the foam insert 102 may be formed of a compliant hydrophilic biocompatible material such as polyurethane foam. The material preferably possesses sponge-like qualities that allow retention within the foam insert 102 of a large volume of fluid 120 while minimizing leakage therefrom.

The shape of the foam insert 102 is preferably tailored to bring the portions of the foam insert 102 saturated with the fluid 120 into contact only with desired portions of the lining 108 of the uterus which portions include target tissue to be treated. For example, the foam insert 102 may be shaped so that contacting portions 118 of an outer surface thereof touch target portions of the uterus lining 108 while other portions 119 of the outer surface of the foam insert 102 are spaced from the lining 108. In the exemplary embodiment, the foam insert 102 is placed into the uterus 106 via the vaginal canal 114 and the cervical opening 110. As would be understood by those skilled in the art, this procedure may be carried out minimally invasively using a hysteroscope to minimize discomfort and complications. The foam insert 102 may be delivered in place within the uterus 106 via a tube 112 that is inserted through the cervical opening 110.

Prior to the insertion, the foam insert 102 is compressed into a small insertion shape that allows it to fit through the tube 112. After being pushed out of the tube 112, the foam insert 102 expands to the operative shape shown in FIG. 1, so that the contacting portions 118 rest against the target portions of the uterine lining 108. As would be understood by those skilled in the art, the foam insert 102 may, for example, be expanded from the insertion shape to the operative shape through compliant properties of the fluid retaining material 104 which cause the fluid retaining material 104 to “pop” into the operative shape once it is no longer constrained within the delivery tube 112.

The shape of the foam insert 102 may also change as the fluid 120 saturates the fluid retaining material 104. For example, soaking the fluid retaining material 104 with saline solution may substantially increase the volume of the foam insert 102 while a certain amount of deformation may also take place as the material of the foam insert 102 becomes saturated with saline. The shape and construction of the dry foam insert 102 is therefore selected to compensate for changes that take place as saline is added to the fluid retaining material 104 so that the shape, once saturated, is the desired operative shape with dimensions selected to place the first portions 118 of the outer surface of the foam insert 102 in contact with the target portions of tissue.

The fluid retaining material 104 of the foam insert 102 is selected to ensure that the fluid 120 is distributed substantially uniformly through the foam insert 102, or more specifically, substantially uniformly through the contacting portions 118. A substantially uniform distribution of the fluid 120 promotes uniformity in the depth of the lesions formed in the portions of the uterine lining 108 adjacent to the contacting portions 118. As described above, the fluid may be a saline solution, or any other biocompatible fluid with appropriate electrically conductive properties. The saline solution may conductivity, so any open-celled foam, e.g., PVA foam, may be utilized with the present invention. The saline is received within the foam insert 102 and help at the desired locations (i.e., the contacting portions 118) during the ablation procedure, rather than flowing freely within the uterine cavity 106. As a result, less fluid 120 is used as compared to procedures where the fluid is free flowing and the procedure may be completed more rapidly. While the system according to the present invention retains most of the saline within a confined volume, it places the fluid in contact with target tissue more effectively than is possible with conventional ablation methods that use fluids confined in bags or tubes.

The efficiency of the system 100 may be enhanced by including an energy receptor within the material of the foam insert 102. For example, as shown in FIG. 2, the foam insert 102 may include an antenna 152 embedded within the fluid retaining material 104 by, for example, insert molding. The shape of the antenna 152 is preferably optimized to obtain a substantially uniform energy distribution throughout the contacting portions 118. In a different embodiment, a material acting as an energy receptor may be seeded in the fluid retaining material 104. For example, small pieces of RF responsive material may be embedded in the foam insert 102, to act as a plurality of small antennas throughout the device. The distribution of the energy receptor throughout the foam insert 102 may also be selected to control the energy distribution therein.

In addition, an optional handle 150 may be formed in the foam insert 102, to facilitate manipulation of the foam insert 102 during insertion into the target body cavity. For example, the antenna 152 may include an elongated component extending out of the foam insert 102 that forms the handle 150. As would be understood by those skilled in the art, the handle 150 is preferably sized and shaped for handling by a physician to position the foam insert 102 in a desired location within the uterus 106. Alternatively, a separate handle may be embedded in the material of the foam insert 102, or may be attached thereon prior to placement of the device.

According to a first embodiment of the invention, the fluid retaining material is exposed as an outer surface of the foam insert 102 at least at the contacting portions 118 to place the fluid 120 in direct contact with the inner lining 108 of the uterus 106. However, in a different embodiment, a lining or outer lining 122 may be applied to the surface 118 to achieve certain results. For example, the lining 122 may enhance the durability of the foam insert 102. The lining 122 may also confer other desirable properties to the foam insert 102. For example, the lining 122 may enhance the lubricity of the foam insert 102 to facilitate insertion into a body cavity or medical compounds may be included in the lining 122 to aid in the treatment of the target tissue, to aid in the healing of the tissue surrounding the treated tissue, or to protect non-targeted tissue. Additives, e.g., surfactants, may be utilized in the foam-making process to regulate pore size and make the outer lining 122 porous when the foam piece is molded.

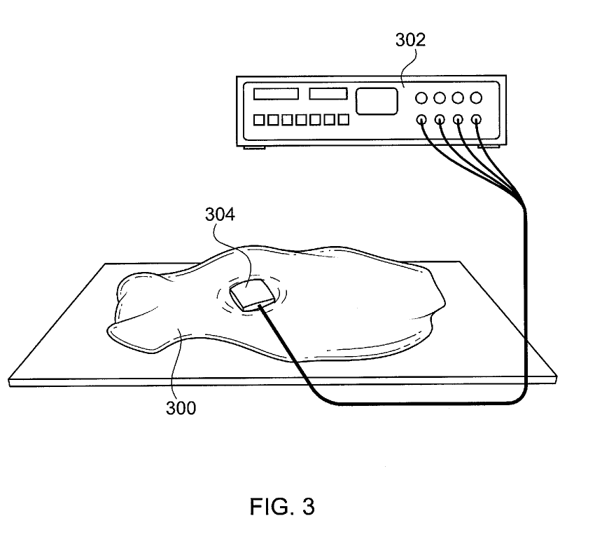

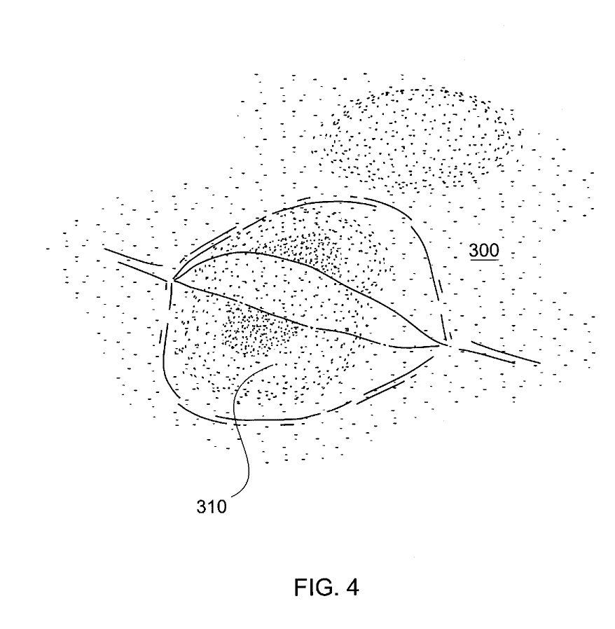

An exemplary embodiment of a foam insert 304 according to the present invention was tested on a slab of animal tissue (beef liver) to determine its effectiveness as an RF electrode. FIG. 3 shows a pictorial representation of the experimental setup used. The fluid retaining material forming the foam insert 304 is polyurethane foam (hydrophilic) which has a high capacity for retaining fluids. When soaked, the foam insert 304 increases its size by about 125% to 130%, and holds approximately 4 times its initial volume in fluid. In this example, the fluid was a saline solution. The foam insert 304 was initially a 5 cm by 5 cm square approximately 2 cm thick, which swelled to 6.5 cm by 6.5 cm after soaking in the saline. The foam insert 304 was placed on the surface of a beef liver 300 and connected to an RF generator 302 after which RF energy was applied in a series of sessions over a given period of time. The duration, power level and impedance found in an initial run representing an exemplary ablation procedure were as follows:

| time (min.) | Power (W) | Impedance (Ω) |
|---|---|---|
| 0 | 10 | 104 |
| 1 | 20 | 95 |
| 2 | 30 | 86 |
| 3 | 40 | 77 |
| 4 | 50 | 74 |
| 5 | 60 | 70 |
| 6 | 80 | 67 |

### After about 6 minutes, the ablation was allowed to continue, which resulted in a large and deep ablation region 310 on the liver 300 which was more substantial than desired.

A subsequent test run was carried out to build on the results described above. In this run, a round foam insert was used, and the power was initially set to a high level of about 80 Watts. The device was run for 3 minutes, followed by 1 minute and then by 2 minutes runs. The shape and the size of the foam insert 304 were also optimized, by using a circular shape, and later by reducing the size of the electrode. The ablation region remained circular in these latter tests, and the size and depth of the lesion increased.

The present invention has been described with reference to specific exemplary embodiments. Those skilled in the art will understand that changes may be made in details, particularly in matters of shape, size, material and arrangement of parts. Accordingly, various modifications and changes may be made to the embodiments. For example, the exemplary devices described may be used to perform ablations of the lining in other bodily cavities or hollow organs other than the uterus. The specifications and drawings are, therefore, to be regarded in an illustrative rather than a restrictive sense.

1. A system for tissue treating tissue within a hollow organ, comprising:
a foam body having an operative shape adapted to fill at least a portion of the hollow organ, the foam body including a fluid retaining portion formed of a material selected to retain therein an electrically conductive fluid, the fluid retaining component including at least one contacting portion which, when the foam body is inserted into the hollow organ in a desired position, contacts a target portion of tissue placing the fluid in contact with selected portions of an inner surface of the hollow organ when the foam body is in the operative shape.

2. The system of claim 1, further comprising:
an energy receptor disposed within the foam body to distribute electrical energy to the contacting portion via the fluid retained within the fluid retaining portion.

3. The system of claim 1, wherein the foam body is substantially smaller in an insertion configuration than in an operative configuration into which it is expanded once inside the hollow organ.

4. The system of claim 1, further comprising:
an insertion tube through which the foam body is inserted into the hollow organ when in the insertion configuration.

5. The system of claim 1, further comprising:
a handle embedded in the foam body.

6. The system of claim 2, wherein the energy receptor is an electrically conductive antenna embedded in the foam body.

7. The system of claim 6, wherein the antenna forms a handle for manipulating the foam body.

8. The system of claim 1, wherein the fluid comprises saline.

9. The system of claim 1, wherein the fluid retaining component comprises a compliant hydrophilic biocompatible material.

10. The system of claim 1, wherein the foam body is sized and shaped for use in a procedure for ablating portions of a lining of a uterus.

11. The system of claim 1, wherein the foam insert includes a plurality of contacting portions and wherein the fluid retaining component provides a substantially uniform distribution of the fluid over the contacting portions.

12. The system of claim 1, wherein the foam body expands from the insertion configuration to the operative configuration when a constraining force is removed therefrom.

13. The system of claim 1, further comprising:
a lining covering a portion of an outer surface of the foam body.

14. The system of claim 1, wherein the foam insert further comprises an electrically conducting contact for coupling the foam insert to an RF generator.

15. A system for ablating a lining of a uterus, comprising:
a foam body adapted to substantially fill at least a portion of the uterus when in an operative configuration so that a contacting portion of an outer surface of the foam body contacts a target portion of the lining to be ablated, the foam body including an energy receiving contact and an electrically conductive fluid which, when provided to the foam body, saturates the foam body to conduct RF energy from the energy receiving contact to a contacting portion of the foam body which, when the foam body is in the operative configuration, is in contact with a target portion of the lining to be ablated.

16. The system of claim 15, further comprising:
an energy receptor within the foam body delivering energy from the energy receiving contact to the contacting portion via the fluid.

17. The system of claim 15, further comprising:
an elongated tube through which the foam body is inserted into the uterus while in the insertion configuration.

18. The system of claim 15, wherein the electrically conductive fluid comprises saline.

19. The system of claim 16, wherein the energy receptor comprises an electrically conductive material embedded within the foam body.

20. The system of claim 20, wherein the electrically conductive material is formed as an antenna embedded in the foam body.

21. The system of claim 20, wherein the antenna forms a handle for manipulating the foam body.

22. The system of claim 15, further comprising:
an RF generator coupled to the energy receiving contact.

23. The system of claim 15, wherein the foam body expands from the insertion configuration to an operative configuration after release from the elongated tube.

24. The system of claim 15, wherein the foam body is formed of a compliant, hydrophilic, biocompatible, sponge material.

25. A method for ablating tissue in a body cavity, comprising:
inserting into the body cavity a foam body in an insertion configuration;
expanding the foam body to an operative configuration within the body cavity so that the foam body substantially fills at least a portion of the cavity with a contacting portion of the foam body contacting a target portion of tissue to be ablated;
permeating the foam body with an electrically conductive fluid; and
transferring energy through the foam body to the contacting portion via the fluid to ablate the target tissue.

26. The method of claim 25, wherein the foam body is inserted into the cavity via an elongated tube.

27. The method of claim 25, wherein the electrically conductive fluid comprises saline.

28. The method of claim 25, wherein the energy transferred through the foam body is RF energy.

29. The method of claim 25, wherein the foam body expands from the insertion configuration to the operative configuration when released from the elongated tube.

30. The method of claim 25, wherein the foam body includes an energy receptor antenna embedded therein.

31. The method of claim 30, wherein the antenna forms a handle for manipulating the foam body.
